(19) United States
(12) Patent Application Publication
(10) Pub. No.: US 2024/0358291 A1
(43) Pub. Date: Oct. 31, 2024

(54) MEDICAL SENSOR

(71) Applicant: Covidien LP, Mansfield, MA (US)

(72) Inventors: Sarah L. Hayman, Boulder, CO (US); Devon E. Scott-Drechsel, Superior, CO (US); Liana J. Kerr-Layton, Boulder, CO (US); Mia D. Fox, Boulder, CO (US); Nicholas J. Vaver, Lafayette, CO (US); Catalina S. Bastías Díaz, Boulder, CO (US); Kathleen C. Stewart, Aurora, CO (US)

(21) Appl. No.: 18/630,426

(22) Filed: Apr. 9, 2024

Related U.S. Application Data
(60) Provisional application No. 63/498,728, filed on Apr. 27, 2023.

(51) Int. Cl. A61B 5/1455; A61B 5/00; A61B 90/00

(52) U.S. Cl. CPC A61B 5/14552; A61B 5/6814; A61B 5/6833; A61B 5/68335; A61B 90/08; A61B 2090/0807; A61B 2562/164

(57) ABSTRACT

In some examples, a kit includes a pulse oximetry sensor patch and a stretchable pressure backing configured to be applied to a region of a patient's skin. An assembly may include the stretchable pressure backing securing the pulse oximetry sensor patch against the patient's skin. The stretchable pressure backing is configured to stretch across the pulse oximetry sensor patch from an unstretched length to a stretched length. When in its stretched length, the stretchable pressure backing is configured to press the pulse oximetry sensor patch against the patient's skin to apply pressure to reduce venous pulsations in the region of the patient's skin. An example technique includes applying the pulse oximetry sensor patch to the patient's skin and stretching the stretchable pressure backing across the pulse oximetry sensor patch to its stretched length to apply pressure to the pulse oximetry sensor patch to reduce venous pulsations.

### CROSS-REFERENCE TO RELATED APPLICATION

The present application claims the benefit of U.S. Provisional Patent Application Ser. No. 63/498,728, filed on Apr. 27, 2023, the entire content of which is incorporated herein by reference.

### TECHNICAL FIELD

The present disclosure generally relates to medical sensors.

### BACKGROUND

Medical sensors are used to monitor one or more physiological parameters of a patient. For example, an oxygen saturation sensor, such as a pulse oximetry sensor or a regional oximetry sensor, is configured to monitor the oxygen saturation levels of a patient. In some examples, a noninvasive pulse oximetry sensor is placed on a patient to measure the oxygen saturation level of the patient via photoplethysmography. When the oxygen saturation level of the patient decreases to reach a desaturation threshold, the oxygen saturation monitoring system may output an indication that the patient is experiencing oxygen desaturation.

### SUMMARY

The present disclosure describes example devices, kits, assemblies, and techniques for monitoring oxygen saturation of a subject.

An example assembly according to aspects of the present disclosure includes a pulse oximetry sensor patch configured to be applied to a region of a patient's skin. The assembly further includes a stretchable pressure backing configured to secure the pulse oximetry sensor patch against the patient's skin. The stretchable pressure backing is configured to stretch across the pulse oximetry sensor patch from an unstretched length to a stretched length. When the stretchable pressure backing is at its stretched length, the stretchable pressure backing is configured to press the pulse oximetry sensor patch against the patient's skin to apply pressure to reduce venous pulsations in the region of the patient's skin.

An example method according to aspects of the present disclosure includes applying a pulse oximetry sensor patch to a region of a patient's skin. The method further includes positioning a stretchable pressure backing across the pulse oximetry sensor patch. The method further includes stretching the stretchable pressure backing across the pulse oximetry sensor patch from an unstretched length to a stretched length to cause the pulse oximetry sensor patch to apply pressure against the patient's skin to reduce venous pulsations in the region of the patient's skin.

A kit according to aspects of the present disclosure includes a pulse oximetry sensor patch configured to be applied to a region of a patient's skin. The kit further includes at least one stretchable pressure backing configured to secure the pulse oximetry sensor patch against the patient's skin. The at least one stretchable pressure backing is configured to stretch across the pulse oximetry sensor patch from an unstretched length to a stretched length. When the stretchable pressure backing is stretched to its stretched length, the stretchable pressure backing is configured to press the pulse oximetry sensor patch against the patient's skin to apply pressure to reduce venous pulsations in the region of the patient's skin.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 a perspective view of an example system configured to monitor one or more physiological parameters of a patient, including an example pulse oximetry sensor patch.

FIG. 2 is a top view of an example kit including a pulse oximetry sensor patch and a stretchable pressure backing.

FIG. 3A is a top view of an initial configuration of an example assembly including the pulse oximetry sensor patch of FIG. 2 applied to a patient's skin and the stretchable pressure backing ready to be stretched over the pulse oximetry sensor patch.

FIG. 3B is a top view of a final configuration of the assembly of FIG. 3A with the stretchable pressure backing stretched and applying pressure on the pulse oximetry sensor patch.

FIG. 4 illustrates the assembly of FIG. 3B applied on a forehead of a patient in an example application.

FIG. 5A is an exploded side view of an example assembly including a pulse oximetry sensor patch and a stretchable pressure backing including an adhesive layer and a release liner.

FIG. 5B is a bottom view of the assembly of FIG. 5A.

FIG. 6A is an exploded side view of an example assembly including a pulse oximetry sensor patch and a stretchable pressure backing including a discontinuous adhesive layer and a release liner.

FIG. 6B is a bottom view of the assembly of FIG. 6A.

FIG. 7A is an exploded side view of an example assembly including a pulse oximetry sensor patch and a stretchable pressure backing including two layers.

FIG. 7B is a bottom view of the assembly of FIG. 7A.

FIG. 8 is a bottom view of an example pulse oximetry sensor patch further including a padding layer.

FIG. 9 is a bottom view of an example pulse oximetry sensor patch further including a padding ring.

FIG. 10 is a flowchart illustrating an example technique of securing a pulse oximetry sensor patch to a patient's skin with a stretchable pressure backing for reducing venous pulsing.

### DETAILED DESCRIPTION

In general, aspects of the present disclosure relate to devices, kits, assemblies, and techniques for monitoring a physiological parameter, such as, but not limited to, oxygen saturation, of a subject.

Optical sensors may be attached to a patient, e.g., on a skin surface, to sense one or more patient parameters. For example, optical sensors may be used to sense temperature, blood pressure, blood flow, heart rate, respiration, or oxygen saturation. The present disclosure describes kits, assemblies, and techniques with reference to pulse oximeter sensors. However, kits, assemblies, and techniques according to the present disclosure may be used with any suitable optical sensor configured to generate a signal indicative of a physiological parameter, such as, but not limited to, a regional oxygen saturation sensor.

A pulse oximetry sensor may be applied to a region of a patient's skin for monitoring oxygen saturation. Certain monitoring regions or sites, for example, a patient's forehead, may exhibit pooling or pulsing of venous blood, which may interfere with the monitoring. For example, a pulse that is synchronous with the right side of the heart may be superimposed on signals detected by the sensor, leading to an additional waveform or wave component. Such an additional waveform, wave component, pulsing, or pooling may result in inaccurate oxygen saturation readings.

Pulse oximeters may be used to non-invasively monitor oxygen saturation, by detecting light transmitted through a target region. A pulse oximeter includes a pulse oximetry sensor configured to sense and send signals to a control circuitry. For example, the pulse oximetry sensor may include one or more photoemitters configured to emit light in a predetermined wavelength range, and one or more detectors sense light from the photoemitter transmitted through the patient's skin. The pulse oximetry sensor may send signals indicative of characteristics of the light emitted by the photoemitter that is received by the detector, and of detected light. The control circuitry may receive the signals and determine oxygen saturation or other patient parameters based on the signals. The pulse oximetry sensor may be applied in contact with a region of a patient's skin, for example, as a patch that secures or houses the photoemitter and detector. Thus, a pulse oximetry sensor patch may detect oxygen saturation in blood flowing through the region.

Pulse oximetry sensor patches may be applied on or about a finger, or a portion of an extremity of the patient. Some patients may exhibit relatively poor perfusion at extremities, and pulse oximetry sensors may be applied at other locations, for example, on foreheads. A forehead pulse oximetry sensor patch may, for example, be applied directly above the left or right eye. Due to the proximity of such a location to the brain, a sensor applied on the forehead may provide relatively faster measurement of oxygen saturation than a sensor applied to a finger (or extremity), and may still measure oxygen saturation when a patient's extremities are poorly perfused. The light path may differ depending on the location of the sensor. For example, transmitted light may be sensed when the sensor is positioned on or about the finger or the portion of the extremity, while reflected light may be sensed when the sensor is positioned on the forehead.

In some cases, accuracy of forehead oxygen saturation monitoring may be adversely impacted when the patient is laying at or near supine (flat, zero degrees) or in a Trendelenburg position (negative 15-30 degrees, with the head inclined downward). In such positions, venous blood may tend to pool in forehead tissue (for example, in the supraorbital vein), and a pulse, synchronous with the right side of the heart, may arise in venous blood. Thus, a signal detected by a pulse oximetry sensor in such a location may include two additive waveforms (arterial and venous), and the oxygen saturation readings may not be as indicative of arterial oxygen saturation as when the patient is in another posture.

Applying pressure over the pulse oximetry sensor (or other optical sensor) may at least partially force venous blood from a sensed tissue volume, reducing or substantially removing the venous waveform, and without substantially affecting the arterial waveform, from the detected signal (or another signal). Thus, applying pressure may improve accuracy of oxygen saturation measurement. For example, an elastic headband may be applied over a pulse oximetry sensor to apply pressure and reduce venous pulsations. However, applying relatively greater pressure may result in patient discomfort, for example, by pressing or tightening of the sensor against the patient's skin. Because venous blood is at a lower pressure than arterial pressure, a relatively low pressure may be sufficient to drive out venous blood from a region, but without driving out arterial blood.

While using a headband may provide a higher accuracy, the headband may be relatively difficult to maneuver and apply, while attaining a target pressure. For example, a clinician may need to lift the patient's head off a resting surface while a strap or another portion of the headband is wrapped underneath the head. A clinician may also need to cinch the headband to a proper position over the sensor. Even if positioned correctly, the headband may slip out of position in response to the patient's movements, for example, head movements. A clinician may overtighten the headband, which may force arterial blood away from the tissue, or insufficiently tighten the headband, which may not adequately remove the influence of venous blood, ultimately resulting in inaccurate oxygen saturation measurements. Components of sensors facing the patient's skin, for example, emitters, detectors, lenses, or other components, may press against the skin, which may adversely impact patient comfort. The headband may slide away from a target position, for example, if improperly secured, or if sweating or accumulation of fluids results in slipping of the headband against skin.

In examples described herein, an assembly includes a pulse oximetry sensor patch configured to be applied to a region of a patient's skin and configured to position a pulse oximetry sensor (or other optical sensor) at a target location on a patient without the need for a headband. The assembly further includes a stretchable pressure backing configured to secure the pulse oximetry sensor patch against the patient's skin. The stretchable pressure backing is configured to stretch across the pulse oximetry sensor patch from an unstretched length to a stretched length. When at the stretched length, the stretchable pressure backing is configured to press the pulse oximetry sensor patch against the patient's skin to apply pressure to reduce venous pulsations in the region of the patient's skin.

Assemblies according to the present disclosure may reduce or substantially remove interference from venous pulsations, even when a patient is in supine or Trendelenburg positions. Assemblies according to the present disclosure may also facilitate applying sensor patches while avoiding undue pressure, and increasing patient comfort. Using the stretchable pressure backing may reduce maneuvering or readjustment of patches, or moving or manipulation of the patient's head or anatomy. The stretchable pressure backing may also promote retaining the patch in a target region, for example, by reducing or avoiding migration away from the target region. The stretchable pressure backing may include an adhesive to facilitate attachment to skin. Further, the stretchable pressure backing may facilitate reducing ambient light interference.

The assemblies may be repositionable, or reusable, for example, by periodically replacing the stretchable pressure backing with a fresh unit, while reusing the sensor patch. Such reuse of the sensor patch may reduce overall costs to medical clinics or other users. Reusing sensor patch may also simplify monitoring by reducing the need to reconnect replacement sensor patches to the sensing system, because replacing the pressure backing would not affect the signal pathway or electrical connection between the sensor patch and the sensing system.

While the present disclosure describes kits, assemblies, and techniques with reference to a stretchable pressure backing and a pulse oximetry sensor patch, any other suitable sensor configuration may be used with backings according to the present disclosure. For example, instead of patches, sensors may be secured to or carried on or within housings, rigid substrates, flexible substrates, bands, rods, or any other sensor devices.

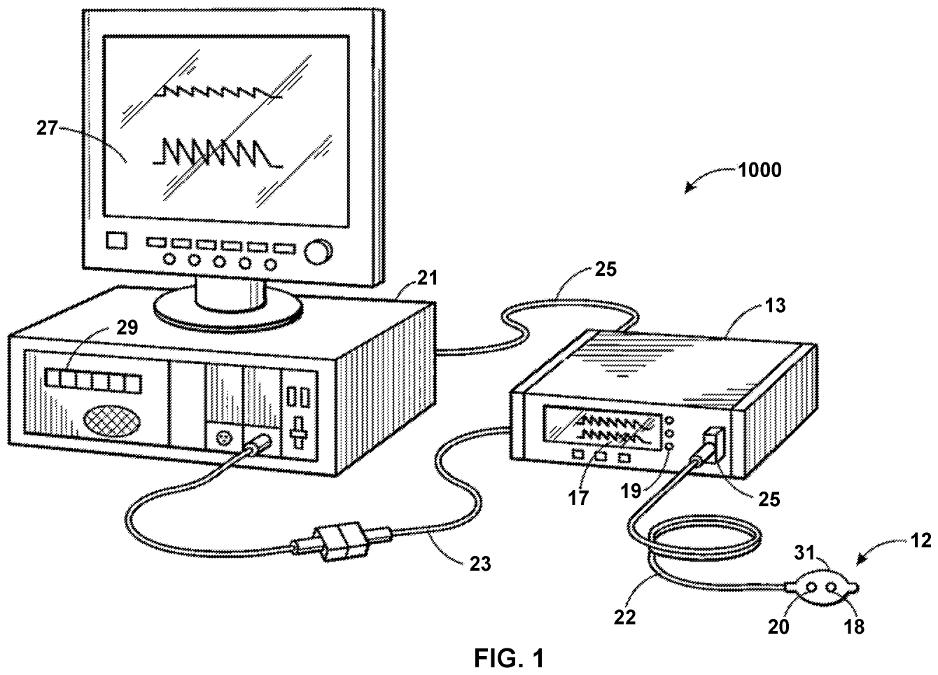

FIG. 1 a perspective view of an example system 1000 configured to monitor one or more physiological parameters of a patient, including an example pulse oximetry sensor patch 12. Pulse oximetry sensor patch 12 may be a forehead patch configured to be applied to the forehead of a patient.

Pulse oximetry sensor patch 12 may represent a MAXFAST™ or other pulse oximetry sensor available from Nellcor Puritan Bennett, LLC. While the present disclosure describes sensors for use on a patient's forehead and/or temple, patch 12 may be configured for use on other tissue locations, such as the finger, the toes, the heel, the ear, or any other appropriate measurement site. While system 1000 illustrated in FIG. 1 relates to photoplethysmography or pulse oximetry, system 1000 may be configured to obtain a variety of medical measurements with a suitable medical sensor. For example, system 1000 may additionally or alternatively be configured to perform regional oximetry, determine patient electroencephalography (e.g., a bispectral index), or any other physiological parameter.

System 1000 includes pulse oximetry sensor patch 12, which may be communicatively coupled to a patient monitor 13. Pulse oximetry sensor patch 12 may include one or more emitters 18 and one or more detectors 20. Pulse oximetry sensor patch 12 may be coupled to the monitor 13 via a cable 22. Cable 22 is configured to interface with the patient monitor 13 through a connector 25, which is adapted to couple to a sensor port of the patient monitor 13. Cable 22 may include a plurality of conductors, such as a first set for emitter 18 and a second set for detector 20, which are configured to carry signals (e.g., electrical signals, optical signals) between patient monitor 13 and pulse oximetry sensor patch 12. The conductors may be surrounded by an insulting material, such that cable 22 is a rounded cable. Alternatively, cable 22 may be a ribbon cable or a flexible circuit cable having a relatively flat profile.

Patient monitor 13 may include a monitor display 17 configured to display information relating to one or more physiological parameters of the patient, information about system 1000, and/or alarm indications. Patient monitor 13 may include various input components 19, such as knobs, switches, keys and keypads, buttons, etc., to provide for operation and configuration of monitor 13. Patient monitor 13 also includes a processor that may be used to execute code such as code for performing diagnostics on system 1000, for measuring and analyzing patient physiological parameters, and so forth.

Patient monitor 13 may be any suitable monitor, such as a pulse oximetry monitor available from Nellcor Puritan Bennett LLC. Furthermore, for providing additional functions, patient monitor 13 may be coupled to a multi-parameter patient monitor 21 via a cable 23 connected to a sensor input port or via a cable 25 connected to a digital communication port. In addition to monitor 13, or alternatively, multi-parameter patient monitor 21 may be configured to calculate physiological parameters and to provide a central display 27 for the visualization of information from patient monitor 13 and from other medical monitoring devices or systems. Multi-parameter monitor 21 includes a processor that may be configured to execute code. Multi-parameter monitor 21 may also include various input components 29, such as knobs, switches, keys and keypads, buttons, etc., to provide for operation and configuration of multi-parameter monitor 21. In addition, patient monitor 13 and/or the multi-parameter monitor 21 may be connected to a network to enable the sharing of information, such as patient physiological data captured by pulse oximetry sensor patch 12, with servers or other workstations.

Pulse oximetry sensor patch 12 may include a sensor body 31 housing one or more optical components. Sensor body 31 may be formed from any suitable material, including rigid or conformable materials, such as foam or other padding materials (e.g., a sponge or gel), fiber, fabric, paper, rubber or elastomeric compositions (including acrylic elastomers, polyimide, silicones, silicone rubber, celluloid, PMDS elastomer, polyurethane, polypropylene, polyethylene, acrylics, nitrile, PVC films, acetates, and latex).

Sensor body 31 may house a number of components, each providing certain functionality. For example, pulse oximetry sensor patch 12 may be a wireless sensor. In such examples, pulse oximetry sensor patch 12 may include a wireless module for establishing a wireless communication with patient monitor 13 and/or the multi-parameter patient monitor 21 using any suitable wireless standard. By way of example, the wireless module may be capable of communicating using one or more of the ZigBee standard, WirelessHART standard, Bluetooth standard, IEEE 802.11x standards, or MiWi standard.

Emitter 18 and detector 20 may be arranged in a reflectance or transmission-type configuration with respect to one another. For example, when configured for use on a patient's forehead, emitter 18 and detector 20 may be in a reflectance configuration. Light from emitter 18 may be used to measure, for example, oxygen saturation, water fractions, hematocrit, or other parameters of a patient. The term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In some examples, detector 20 may include an array of detector elements that may be capable of detecting light at various intensities and wavelengths. Light may enter detector 20 after passing through the tissue of the patient. Alternatively, light emitted from the emitter 18 may be reflected by elements in the tissue of the patient to enter detector 20. Detector 20 may convert the received light at a given intensity, which may be directly related to the absorbance and/or reflectance of light in the tissue of the patient, into an electrical signal. That is, when more light at a certain wavelength is absorbed, less light of that wavelength is typically received from the tissue by detector 20, and when more light at a certain wavelength is reflected, more light of that wavelength is typically received from the tissue by detector 20. After converting the received light to an electrical signal, detector 20 may send the signal to monitor 13, where physiological characteristics may be calculated based at least in part on the absorption and/or reflection of light by the tissue of the patient.

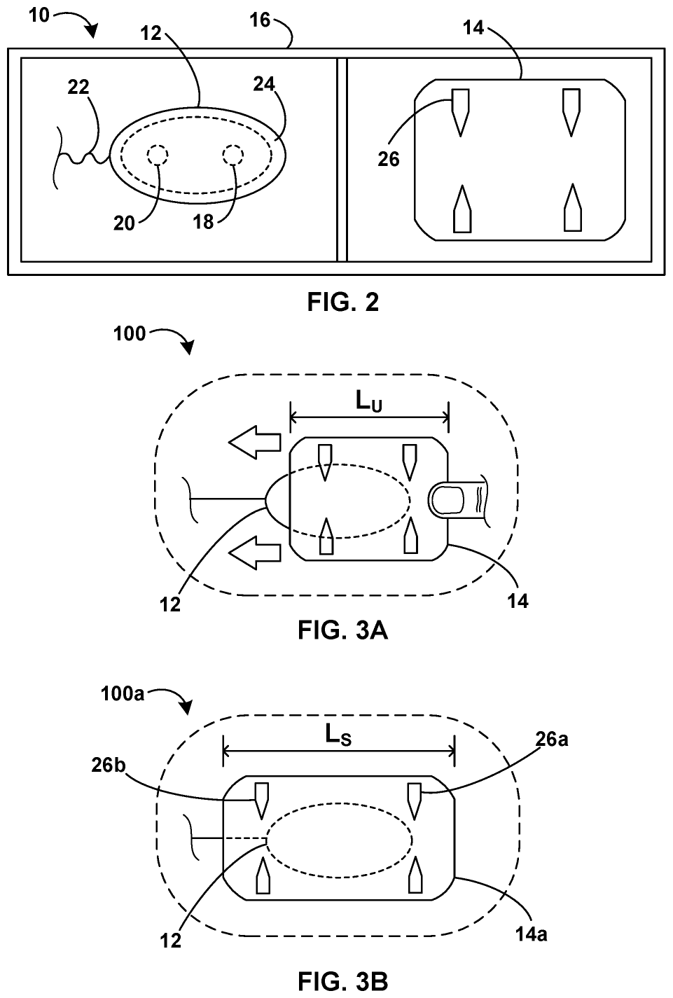

FIG. 2 is a top view of an example kit 10 including a pulse oximetry sensor patch 12 and a stretchable pressure backing 14. Pulse oximetry sensor patch 12 is configured to be applied to skin (or another external surface) of a patient, for example, to monitor oxygen saturation. Stretchable pressure backing 14 is configured to apply pressure to the skin of the patient (for example, being applied over or across pulse oximetry sensor patch 12) to reduce venous pulsations.

In the example shown in FIG. 2, kit 10 includes a housing 16, for example, including a rigid material, a flexible material, or combinations thereof. For example, housing 16 may include a pouch, a box, a bag, a hardboard, a backing sheet, an insert, or combinations thereof. Housing 16 may include paper, plastic, metal, alloy, woven material, nonwoven material or any other suitable packaging material or combinations thereof. Housing 16 may define more than one compartment, or include sub-housings holding one or more units of pulse oximetry sensor patch 12 or of stretchable pressure backing 14. Housing 16, or sub-housings in housing 16, may maintain components therein in a sterile state, for example, prior to opening. For example, an interior of housing 16 may hold a vacuum, or a reduced pressure interior, a gas, or a gaseous mixture. In other examples, the components of kit 10 can be provided to a user using techniques other than or in addition to housing 16.

While kit 10 shown in FIG. 2 includes a single pulse oximetry sensor patch 12 and a single stretchable pressure backing 14, in other examples, kit 10 may include more than one pulse oximetry sensor patch 12 and/or more than one stretchable pressure backing 14. In some examples, kit 10 includes more than one stretchable pressure backing 14 for each pulse oximetry sensor patch 12, such that different units of stretchable pressure backing 14 may be used with each pulse oximetry sensor patch 12. For example, kit 10 may include at least two, at least three, at least four, or at least five stretchable pressure backings 14. Different units of stretchable pressure backing 14 may differ in shape or size. A clinician can, for example, select from the provided stretchable pressure backings 14 based on preference or based on the particular needs of the patient. In other examples, kit 10 includes more than one stretchable pressure backing 14 for each pulse oximetry sensor patch 12, where at least two stretchable pressure backings 14 are substantially similar (e.g., identical but for manufacturing variations). This can provide the clinician with a back-up pressure backing 14, e.g., in case another pressure backing 14 is rendered unusable.

Pulse oximetry sensor patch 12 is configured to be applied to a surface of a patient, which is primarily referred to as skin of the patient but can be another suitable surface, to monitor oxygen saturation or other patient parameters, for example, heart rate or other physiological parameters. Pulse oximetry sensor patch 12 may include at least one emitter 18 configured to emit light through a region or a thickness of a patient's skin, and at least one detector 20 configured to receive light transmitted through the region or the thickness of the patient's skin. Pulse oximetry sensor patch 12 may further include a cable 22 to transmit a signal indicative of sensed light, or any signal indicative of oxygen saturation. The signal may be received by a control circuitry or processing circuitry, for example, in monitors 13 or 21, which in turn may determine the oxygen saturation based on the signal. The signal can be, for example, a photoplethysmogram (PPG) signal or a regional oximetry signal. Instead of, or in addition to, cable 22, pulse oximetry sensor patch 12 may include wireless circuitry configured to send a wireless signal to the control circuitry or the processing circuitry.

The processing circuitry or the control circuitry may include one or more processors, and may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 190 may include multiple components, such as any combination of one or more microprocessors, one or more DSPs, one or more ASICs, or one or more FPGAs, as well as other discrete or integrated logic circuitry, and/or analog circuitry.

In some examples, pulse oximetry sensor patch 12 includes a sensor adhesive layer 24. Sensor adhesive layer 24 may extend over a portion or over an entirety of a surface of pulse oximetry sensor patch 12 configured to contact a patient's skin. In some examples, sensor adhesive layer 24 extends in a peripheral region about a periphery of pulse oximetry sensor patch 12. Sensor adhesive layer 24 may or may not extend across emitter 18 or detector 20. Sensor adhesive layer 24 may be optically transparent or at least partially transmissive, for example, at least in wavelength ranges detected by detector 20, or in other predetermined wavelength ranges. Sensor adhesive layer 24 may include any suitable adhesive. In some examples, sensor adhesive layer 24 may include silicone, acrylic, or combinations thereof.

Stretchable pressure backing 14 is configured to secure the pulse oximetry sensor patch 12 against the patient's skin, while applying pressure to the patient's skin to reduce venous pulsations. For example, stretchable pressure backing 14 may be configured to stretch across pulse oximetry sensor patch 12 from an unstretched length L_{U}, (shown in FIG. 3A) to a stretched length L_{S} (shown in FIG. 3B). When stretchable pressure backing 14 is stretched to its stretched length L_{S}, stretchable pressure backing 14 presses pulse oximetry sensor patch against the patient's skin to apply pressure to reduce venous pulsations in the region of the patient's skin. For example, the applied pressure may vary with the extent of stretching between L_{U} and L_{S}, as described with reference to FIGS. 3A and 3B.

FIG. 3A is a top view of an initial configuration of an example assembly 100 including pulse oximetry sensor patch 12 of FIG. 2 applied to the patient's skin and stretchable pressure backing 14 ready to be stretched over pulse oximetry sensor patch 12. Pulse oximetry sensor patch 12 may be placed on or applied to the patient's skin, for example, in a forehead region, or any other target region. Pulse oximetry sensor patch 12 may be partially secured to the patient's skin, or at least sufficiently secured to remain substantially in place when stretchable pressure backing 14 is stretched. Pulse oximetry sensor patch 12 may initially be held in place by one or more of sensor adhesive layer 24, water, sweat, saline, or another agent, or by friction, capillary forces, or external pressure, for example, applied by a clinician.

In the initial configuration, stretchable pressure backing 14 is substantially unstretched, and has an unstretched length L_{U} in a direction to be stretched. In the initial configuration, the unstretched length L_{U} of stretchable pressure backing 14 may not substantially apply pressure on the patient's skin. The initial configuration can also be referred to as an at-rest position in some examples.

From the initial configuration of FIG. 3A, stretchable pressure backing 14 is stretched in the direction indicated by the double arrows, across a length of pulse oximetry sensor patch 12. As stretchable pressure backing 14 is stretched, increasing pressure is exerted by stretchable pressure backing 14 due to tension arising from the stretching, against pulse oximetry sensor patch 12, and ultimately against the patient's skin.

FIG. 3B is a top view of a final configuration 100a of the assembly of FIG. 2A with stretchable pressure backing 14 stretched to a stretched configuration 14a and applying a target pressure on pulse oximetry sensor patch 12. In the stretched configuration 14a, stretchable pressure backing 14 has a stretched length L_{S}. When stretchable pressure backing 14 is stretched to its stretched length L_{S} and positioned over pulse oximetry sensor patch 12 and over the skin of the patient, stretchable pressure backing 14 applies a target pressure to pulse oximetry sensor patch 12, the target pressure being sufficient to remove venous pulsations in the region to which pulse oximetry sensor patch 12 is applied (or to which stretchable pressure backing 14 is applied). In some examples, the target pressure may not completely remove venous pulsations, but may sufficiently reduce venous pulsations to avoid interference with monitoring of oxygen saturation via pulse oximetry sensor patch 12. The target pressure can be a range of pressure values, which may vary based on the surface area of pulse oximetry sensor patch 12.

While stretchable pressure backing 14 may be stretched in a direction aligned with a major axis or a major dimension of pulse oximetry sensor patch 12, in other examples, stretchable pressure backing 14 may be stretched in any suitable direction, for example, transverse to or angled relative to the major axis or the major dimension of pulse oximetry sensor patch 12.

In some examples, stretchable pressure backing 14 is transparent or translucent at least in a portion thereof, such that a clinician may ascertain positioning of stretchable pressure backing 14 relative to pulse oximetry sensor patch 12. For example, an entirety of stretchable pressure backing 14 may be transparent or translucent, or stretchable pressure backing 14 may define one or more transparent or translucent windows.

In some examples, stretchable pressure backing 14 may be stretchable beyond stretched length L_{S}, so that a clinician may apply additional pressure, or otherwise adjust the amount of pressure exerted by stretchable pressure backing 14. In other examples, stretchable pressure backing 14 may resist stretching beyond stretched length L_{S}, such that the resistance to further stretching provides tactile feedback to the clinician that sufficient stretching (and the corresponding target pressure applied to sensor patch 12) has been achieved. Resistance to stretching beyond stretched length L_{S} may be provided by varying the thickness of stretchable pressure backing 14 in different regions, providing reinforcing elements in certain regions, varying Young's modulus in different regions, varying material density along length, providing one or more stretch-resistant threads or filaments extendible to a predetermined length, or otherwise by varying elastic characteristics of stretchable pressure backing 14, or combinations thereof.

In some examples, instead of, or in addition to, stretching indicia 26, stretchable pressure backing 14 includes a dual-color layering mechanism to indicate the extent of stretching. For example, stretchable pressure backing 14 may include two or more layers with different coloring, and as a top layer stretches, changes in material property or gaps may reveal a different color in another layer underneath the top layer when a desired tension (for example, associated with stretched length L_{S}) has been achieved.

Stretchable pressure backing 14 may include, consist of, or consist essentially of one or more materials. For example, stretchable pressure backing 14 may include a plastic, a cellulosic material, or a fabric. Stretchable pressure backing 14 may include one or more layers including continuous or discontinuous films, woven, or non-woven portions. In examples, stretchable pressure backing 14 includes a non-woven material.

In examples, stretchable pressure backing 14 may include a four-way stretch tape, a foam, an acrylic foam, foam assortment, non-woven acrylic, spunlace non-woven acrylic, spunlace non-woven high-tack silicone, polyurethane, silicone, polyurethane high-tack silicone, or combinations thereof.

In some examples, the stretching of stretchable pressure backing 14 is fully or partially reversible, for example, being reversible at least in lengths from L_{U} to L_{S}. Thus, if released, and in absence of a holding or retention force, stretchable pressure backing 14 may tend to return to unstretched length L_{U}. In other examples, stretching of stretchable pressure backing 14 is irreversible or incompletely reversible beyond length L_{S}. For example, when stretched beyond length L_{S} (or a predetermined fraction beyond L_{S}), stretchable pressure backing 14 may deform plastically instead of elastically, and may not return to an initial unstretched length.

In some examples, the stretched length L_{S} (for example, beginning from the unstretched length L_{U}) is selected such that when stretchable pressure backing 14 is configured to, when stretched to the stretched length L_{S}, apply pressure to the patient's skin in a range from 6 millimeters of mercury (mmHg) to 20 mmHg, in which a range of pressure is sufficient to substantially remove venous pulsations from the region of the patient's skin on which the pressure is applied.

The stretched length L_{S} may be any length sufficient to cause the pressure to be applied, for example, being at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 140%, or at least 150% greater than the unstretched length Lt.

In some examples, stretchable pressure backing 14 further includes one or more stretching indicia 26 indicative of target stretched length L_{S}. For example, stretching indicia 26 may include visual indicia, such as alignment marks (e.g., arrows or lines), configured to align with ends of pulse oximetry sensor patch 12. For example, one or more of alignment marks aligned with one end of pulse oximetry sensor patch 12 in an initial unstretched configuration 14, as shown in FIG. 3A, with additional one or more alignment marks aligned with another end of pulse oximetry sensor patch 12 in a final stretched configuration 14a, as shown in FIG. 3B. For example, stretching indicia 26 may include a first indicia 26a alignable with a first end of pulse oximetry sensor patch 12 and a second indicia 26b alignable with a second end of pulse oximetry sensor patch 12 along stretched length L_{S} of stretchable pressure backing 14.

Thus, a clinician may hold a first end of stretchable pressure backing 14, for example, with a finger or a tool, and pull a second end to cause stretchable pressure backing 14 to stretch, until stretching indicia 26 indicate that sufficient stretching (L_{S}) has been achieved.

The alignment marks 26a, 26b can have any suitable form. For example, the alignment marks 26a, 26b may include printed, embossed, raised, etched, or cut, features or marks, or combinations thereof. The marks 26a, 26b may include letters, words, numbers, alphanumeric symbols, icons, or any suitable graphical symbols. The alignment marks 26a, 26b may be opaque, translucent, or transparent. In some examples, one or more alignment marks 26a, 26b may include windows in stretchable pressure backing 14 revealing portions of patient's skin or pulse oximetry sensor patch 12 under the respective alignment marks.

Thus, stretchable pressure backing 14 may be used to apply pulse oximetry sensor patch 12 to the patient's skin, while applying sufficient pressure to reduce venous pulsations.

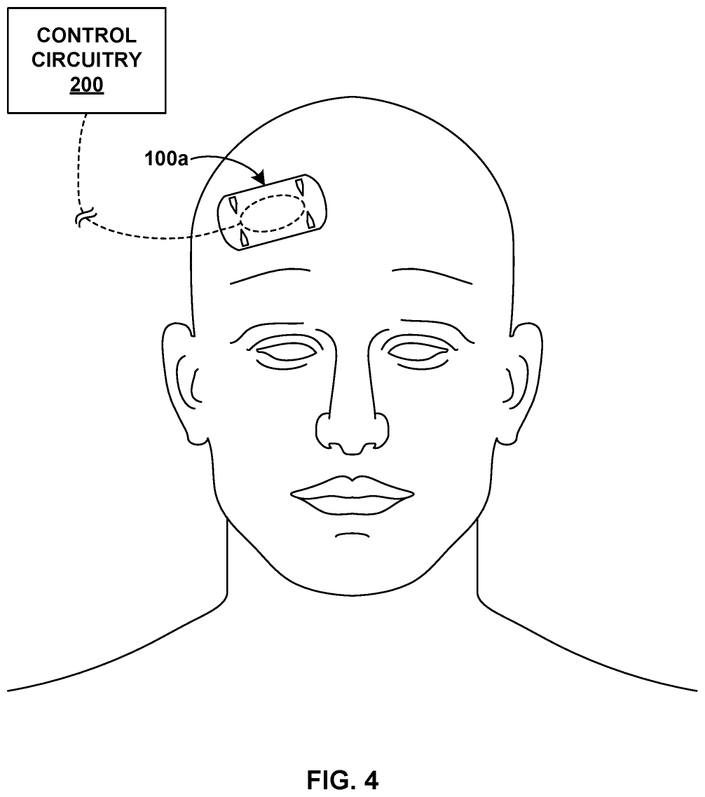

FIG. 4 illustrates the assembly 100a of FIG. 3B applied on a forehead of a patient in an example application. Pulse oximetry sensor patch 12 of assembly 100a may be in wired or wireless communication with control circuitry 200, for example, to transmit a signal indicative of a monitored patient parameter, for example, oxygen saturation.

Control circuitry 200 can include one or more processors, and may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 190 may include multiple components, such as any combination of one or more microprocessors, one or more DSPs, one or more ASICs, or one or more FPGAs, as well as other discrete or integrated logic circuitry, and/or analog circuitry.

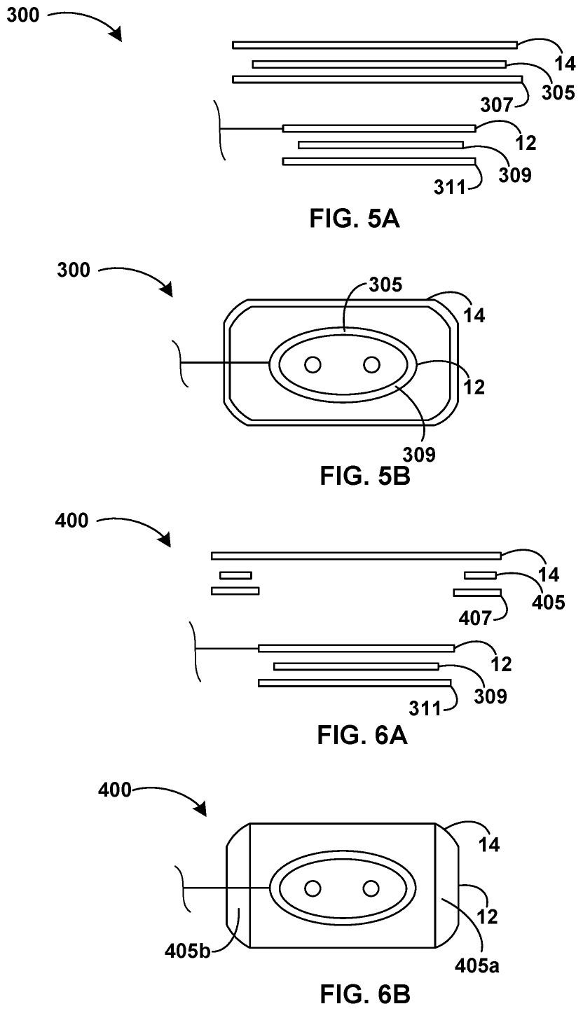

Further examples or pulse oximetry sensor patch 12 or stretchable pressure backing 14 usable in various kits, assemblies, and techniques according to the disclosure are described with reference to FIGS. 5A to 9.

FIG. 5A is an exploded side view of an example assembly 300 including pulse oximetry sensor patch 12 and stretchable pressure backing 14 including an adhesive layer and a release liner. FIG. 5B is a bottom view of assembly 300 of FIG. 5A (without release liner). The adhesive layer or the release liner may be applied over any component of assembly 300. For example, stretchable pressure backing 14 may include a backing adhesive layer 305 configured to secure at least a portion of stretchable pressure backing 14 to a surface (e.g., skin) of a patient. In some examples, backing adhesive layer 305 may be substantially continuous (e.g., continuous or nearly continuous but for manufacturing variations) over an entire surface of stretchable pressure backing 14.

Stretchable pressure backing 14 may further include a backing release liner 307 covering at least a portion of backing adhesive layer 305. For example, a clinician may initially place stretchable pressure backing 14 against the patient's skin with backing release liner 307 in place, to determine a suitable position or orientation, and remove backing release liner 307 to expose backing adhesive layer 305 when stretchable pressure backing 14 is suitably oriented or aligned.

The clinician may initially adhere a portion of backing adhesive layer 305 against the patient's skin or against pulse oximetry sensor patch 12 in an initial unstretched configuration of stretchable pressure backing 14, and progressively contact and adhere increasing portions of backing adhesive layer 305 as stretchable pressure backing 14 is stretched. In other embodiments, the clinician may not progressively contact or adhere backing adhesive layer 305 during stretching, and may instead contact or adhere a portion of backing adhesive layer 305 (or an entirety of backing adhesive layer 305) only after stretchable pressure backing 14 is in stretched configuration 14a (for example, as shown in FIG. 3B) with length L_{S}.

In some examples, pulse oximetry sensor patch 12 further includes a sensor adhesive layer 309 configured to secure pulse oximetry sensor patch 12 to the patient's skin. Pulse oximetry sensor patch 12 may further include sensor release liner 311 covering at least a portion of sensor adhesive layer 309. For example, a clinician may initially position and orient pulse oximetry sensor patch 12 against a region of the patient's skin, and remove sensor release liner 311 to expose sensor adhesive layer 309 and adhere pulse oximetry sensor patch 12 to the patient's skin. For example, the clinician may adhere pulse oximetry sensor patch 12 prior to applying stretchable pressure backing 14.

In other examples, pulse oximetry sensor patch 12 may not include a sensor adhesive layer 309, and may be substantially held in place during (and after) stretching of stretchable pressure backing 14 by the pressure exerted by the stretchable pressure backing 14, optionally in conjunction with backing adhesive layer 305. For example, a portion of backing adhesive layer 305 may contact or surround an outer perimeter of pulse oximetry sensor patch 12, resisting or preventing movement of pulse oximetry sensor patch 12 after stretchable pressure backing 14 is completely applied in a stretched configuration.

One or both of an adhesive layer or a release liner may be discontinuous, or in multiple portions or pieces, as described with reference to FIGS. 6A and 6B.

FIG. 6A is an exploded side view of an example assembly 400 including pulse oximetry sensor patch 12 and stretchable pressure backing 14 including a discontinuous adhesive layer 405 and a release liner 407. FIG. 6B is a bottom view of assembly 400 of FIG. 6A (without a release liner). Discontinuous adhesive layer 405 may include two or more distinct and separate regions, as shown in FIG. 6B, or may include a single region defining a window or discontinuity. Release liner 407 may be continuous and cover multiple regions or separate discontinuous portions or portions of the discontinuous adhesive layer 305, or may be discontinuous, with one or more separately releasable portions. For example, backing adhesive layer 405 may include a first adhesive portion 405a spaced from a second adhesive portion 405b. First adhesive portion 405a and second adhesive portion 405b may be configured to straddle pulse oximetry sensor patch 12.

In some examples, a clinician may remove a portion of release liner 407 to expose and adhere first adhesive portion 405a of discontinuous adhesive layer 405 in an initial unstretched configuration of stretchable pressure backing 14 (optionally leaving second adhesive portion 405b of discontinuous adhesive layer 405 covered), and adhere second adhesive portion 405b in the final stretched configuration 14a of stretchable pressure backing 14. Thus, the clinician may avoid premature attaching of one or more portions of stretchable pressure backing 14 before stretchable pressure backing 14 is stretched to target length L_{S}.

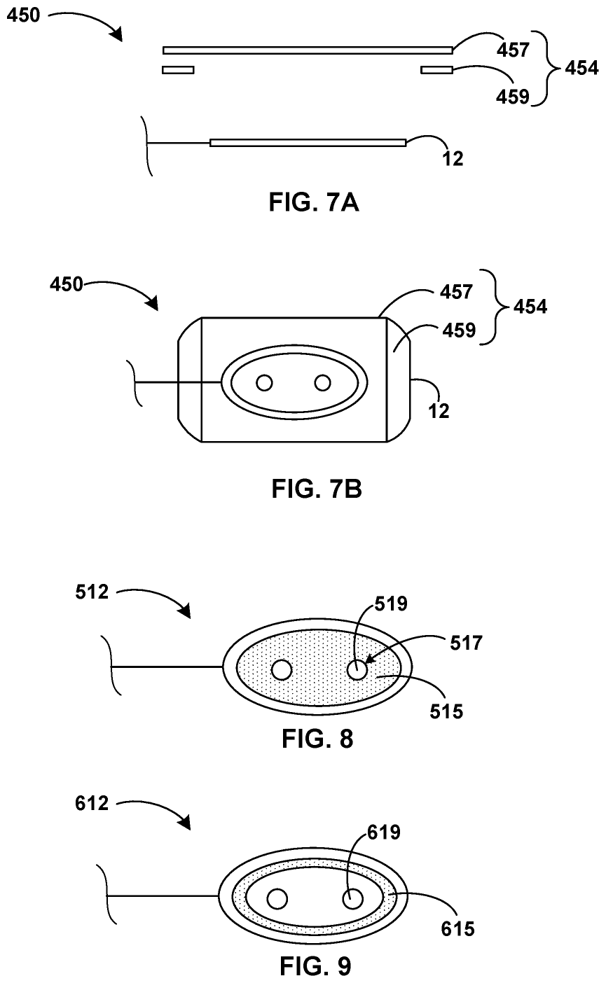

FIG. 7A is an exploded side view of an example assembly 450 including a pulse oximetry sensor patch 12 and a stretchable pressure backing 454 including two layers 457 and 459. FIG. 7B is a bottom view of the assembly of FIG. 7A.

First layer 457 may include an elastic material, while second layer 459 may include a non-clastic material. The clastic material or the non-elastic material may include a foam layer, a woven layer, or a non-woven layer. In some examples, second layer 459 may include a material that has a lower elasticity than the elastic material of first layer 457. Second layer 459 may have a higher stiffness or hardness relative to first layer 457. Second layer 459 may overlay a portion of first layer 457, or may be arranged end-to-end on opposing edges of first 457. Second layer 459 may be adhered, welded, or otherwise secured or attached to an edge or a face of first layer 457.

At least a portion of one or both of first layer 457 or second layer 459 may include an adhesive. In some examples, adhesive is applied only to second layer 459. In some examples, adhesive may be applied to a portion of first layer 457 adjacent second layer 459. In some examples, pulse oximetry sensor patch 12 may overlay only a portion of first layer 457, for example, a portion straddled by second layer 459. In other examples, a portion of pulse oximetry sensor patch 12 may overlay second layer 459. While second layer 459 may be between first layer 457 and pulse oximetry sensor patch 12 as shown in FIG. 7A, in other examples, first layer 457 may be between at least a portion of second layer 459 and pulse oximetry sensor patch 12.

FIG. 8 is a bottom view of an example pulse oximetry sensor patch 512 including a padding layer 515. Pulse oximetry sensor patch 512 is an example of pulse oximetry sensor patch 12 described with reference to FIGS. 2 to 6B. Padding layer 515 extends over a portion of or along an entirety of a surface of pulse oximetry sensor patch 512. Padding layer 515 may be present at a surface of, or embedded within a thickness of, pulse oximetry sensor patch 512. Padding layer 515 may promote patient comfort by reducing or avoiding pressure on patient skin, or discomfort, arising from contact of sensor components (such as emitter 18 or detector 20) with patient's skin. Padding layer 515 may be similar to one or more padding layers described in U.S. Pat. No. 9,138,181, which is incorporated herein in its entirety by reference.

In some examples, padding layer 515 defines at least one sensor window 517 extending across at least one optical component 519 of pulse oximetry sensor patch 512. For example, a padding material may be absent in a region defining sensor window 517.

Padding layer 515 may increase comfort and reduce pressure on the patient's skin in a region of pulse oximetry sensor patch 512 contacting the patient's skin. Padding layer 515 may have any suitable color. In examples, padding layer 515 has a black color, or a relatively dark color, or a color that absorbs light emitted by pulse oximetry sensor patch 512, to absorb stray light not transmitted through the patient's skin. Padding layer 515 may have any suitable thickness. In some examples, padding layer 515 has a thickness of 4 mm or less, for example 2 mm. Without being bound by theory, a thickness of greater than 4 mm may reduce efficacy of sensing oxygen saturation, for example, by interfering with a light path used for monitoring oxygen saturation.

FIG. 9 is a bottom view of an example pulse oximetry sensor patch 612 further including a padding ring 615. Padding ring 615 may be substantially similar to padding layer 515, but formed as a ring. Padding ring 615 may surround at least one optical component 619 of pulse oximetry sensor patch 512.

In some examples, an adhesive, a release liner, or combinations thereof may extend over padding layer 515 or padding ring 615.

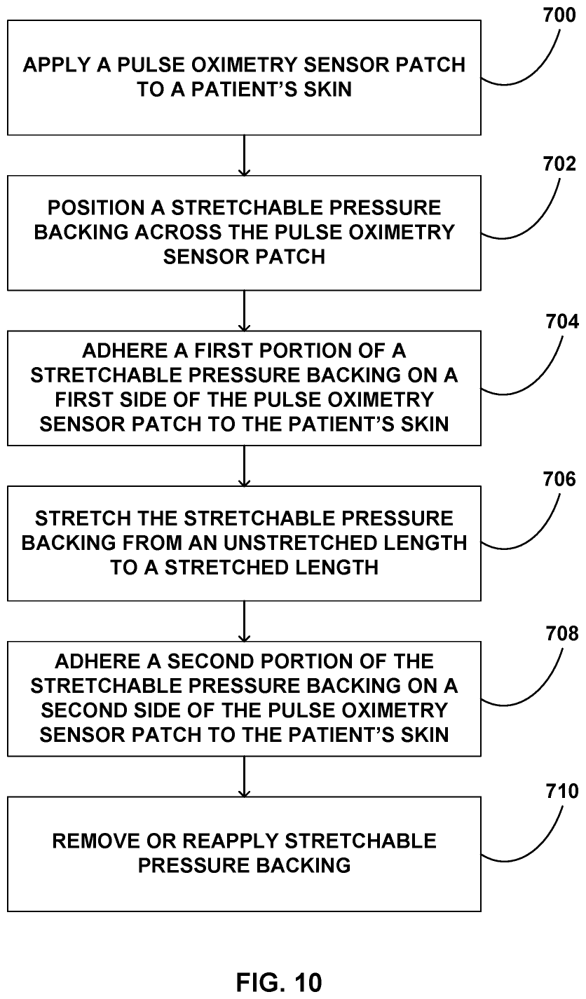

FIG. 10 is a flowchart illustrating an example technique of securing pulse oximetry sensor patch 12 to a patient's skin with stretchable pressure backing 14 to reduce venous pulsations at the target region. While the technique is described with reference to pulse oximetry sensor patch 12 and stretchable pressure backing 14, the technique may be practiced with any pulse oximetry sensor or stretchable pressure backing according to the present disclosure.

The technique includes applying pulse oximetry sensor patch 12 to a region of a patient's skin (700). Prior to the applying (700), material in contact with the skin may be smoothed and checked to promote proper adhesion.

The technique may further include positioning stretchable pressure backing 14 across pulse oximetry sensor patch 12 (702). The technique may further include stretching stretchable pressure backing 14 across the pulse oximetry sensor patch from an unstretched length L_{U} to a stretched length L_{S} (706). When stretchable pressure backing 14 is stretched to its stretched length L_{S}, stretchable pressure backing 14 applies pressure against the patient's skin to reduce venous pulsations in the region of the patient's skin. In some examples, pressure backing 14 is configured such that when pressure backing 14 is at its stretched length and positioned over pulse oximetry sensor patch 12, pressure backing 14 applies pressure in a range from 6 mmHg to 20 mmHg to the skin of the patient. In some examples, the stretched length L_{S} is at least 20% greater than the unstretched length L_{U}.

The technique may further include (optionally) adhering, before the stretching (706), a first portion of stretchable pressure backing 14 on a first side of pulse oximetry sensor patch 12 to the patient's skin (704). The technique may further include (optionally) adhering, after the stretching, a second portion of stretchable pressure backing 14 on a second side of pulse oximetry sensor patch 12 to the patient's skin (708).

After a period of use, stretchable pressure backing 14 may be removed (710), optionally reapplied, or replaced with a fresh unit of stretchable pressure backing 14. For example, pulse oximetry sensor patch 12 may be left in place during the removal and reapplication (710). In other embodiments, pulse oximetry sensor patch 12 may be repositioned by the clinician after removing and prior to replacing stretchable pressure backing 14. Removing stretchable pressure backing 14 (710) may include slowly pulling stretchable pressure backing 14 away from the skin, for example, beginning with one end and progressing to another end of stretchable pressure backing 14. The removal may be sufficiently slow to reduce patient discomfort. In some examples, pulse oximetry sensor patch 12 may be removed from patient's skin after removing stretchable pressure backing 14 (710). In some such examples, the same unit of pulse oximetry sensor patch 12 may be reapplied with a fresh unit of stretchable pressure backing 14 applied over pulse oximetry sensor patch 12.

Various examples of the disclosure have been described. Any combination of the described systems, operations, or functions is contemplated. These and other examples are within the scope of the following claims.

What is claimed is:

1. An assembly comprising:
a pulse oximetry sensor patch configured to be applied to a region of skin of a patient; and
a stretchable pressure backing configured to secure the pulse oximetry sensor patch against the skin,
wherein the stretchable pressure backing is configured to stretch across the pulse oximetry sensor patch from an unstretched length to a stretched length, wherein when the stretchable pressure backing is stretched to the stretched length, the stretchable pressure backing is configured to press the pulse oximetry sensor patch against the skin to apply pressure to reduce venous pulsations in the region of the skin.

2. The assembly of claim 1, wherein the stretched length is configured to apply pressure in a range from 6 millimeters of mercury (mmHg) to 20 mmHg.

3. The assembly of claim 1, wherein the stretched length is at least 20% greater than the unstretched length.

4. The assembly of claim 1, wherein the stretchable pressure backing comprises a backing adhesive layer configured to secure at least a portion of the stretchable pressure backing to the skin.

5. The assembly of claim 4, wherein the backing adhesive layer is substantially continuous over an entire surface of the stretchable pressure backing.

6. The assembly of claim 4, wherein the backing adhesive layer comprises a first adhesive portion spaced from a second adhesive portion, wherein the first adhesive portion and the second adhesive portion are configured to straddle the pulse oximetry sensor patch.

7. The assembly of claim 4, wherein the stretchable pressure backing further comprises a backing release liner covering at least a portion of the backing adhesive layer.

8. The assembly of claim 1, wherein the pulse oximetry sensor patch further comprises a sensor adhesive layer configured to secure the pulse oximetry sensor patch to the skin.

9. The assembly of claim 8, wherein the pulse oximetry sensor patch further comprises a sensor release liner covering at least a portion of the sensor adhesive layer.

10. The assembly of claim 1, wherein the stretchable pressure backing further comprises stretching indicia indicative of a target stretched length.

11. The assembly of claim 10, wherein the stretching indicia comprises a first indicia alignable with a first end of the pulse oximetry sensor patch and a second indicia alignable with a second end of the pulse oximetry sensor patch along the stretched length of the stretchable pressure backing.

12. The assembly of claim 1, wherein the pulse oximetry sensor patch further comprises a padding layer.

13. The assembly of claim 12, wherein the padding layer defines at least one sensor window extending across at least one optical component of the pulse oximetry sensor patch.

14. The assembly of claim 1, wherein the stretchable pressure backing comprises a non-woven material.

15. A method comprising:
applying a pulse oximetry sensor patch to a region of skin of a patient;
positioning a stretchable pressure backing across the pulse oximetry sensor patch; and
stretching the stretchable pressure backing across the pulse oximetry sensor patch from an unstretched length to a stretched length to cause the pulse oximetry sensor patch to apply pressure against the skin to reduce venous pulsations in the region of the skin.

16. The method of claim 15, wherein the stretched length is configured to apply pressure in a range from 6 millimeters of mercury (mmHg) to 20 mmHg.

17. The method of claim 15, wherein the stretched length is at least 20% greater than the unstretched length.

18. The method of claim 15, further comprising:
adhering, before the stretching, a first portion of the stretchable pressure backing on a first side of the pulse oximetry sensor patch to the patient's skin; and
adhering, after the stretching, a second portion of the stretchable pressure backing on a second side of the pulse oximetry sensor patch to the patient's skin.

19. The method of claim 15, wherein the stretching comprises stretching the stretchable pressure backing such that a first stretching indicia of the stretchable pressure backing aligns with a first end of the pulse oximetry sensor patch and a second stretching indicia of the stretchable pressure backing aligns with a second end of the pulse oximetry sensor patch along the stretched length of the stretchable pressure backing.

20. A kit comprising:
a pulse oximetry sensor patch configured to be applied to a region of a skin of a patient; and
at least two stretchable pressure backings configured to secure the pulse oximetry sensor patch against the skin,
wherein each of the stretchable pressure backings is configured to stretch across the pulse oximetry sensor patch from an unstretched length to a stretched length in which the stretchable pressure backing presses the pulse oximetry sensor patch against the patient's skin to apply pressure to reduce venous pulsations in the region of the skin.
